(19) United States
(12) Patent Application Publication
(10) Pub. No.: US 2022/0184060 A1
(43) Pub. Date: Jun. 16, 2022

(54) PHARMACEUTICAL COMPOSITION COMPRISING SPHERICAL AGGLOMERATES OF TIMAPIPRANT

(71) Applicant: Chiesi Farmaceutici S.p.A., Parma (IT)

(72) Inventors: Paolo Simone TISENI, Parma (IT); Marisa PERTILE, Parma (IT)

(21) Appl. No.: 17/421,710

(22) PCT Filed: Dec. 19, 2019
(86) PCT No.: PCT/EP2019/086471

§ 371 Date: Jul. 8, 2021

(30) Foreign Application Priority Data
Dec. 27, 2018 (EP) 18248081.4

(51) Int. Cl. A61K 31/4709; A61K 9/16

(52) U.S. Cl. CPC A61K 31/4709; A61K 9/16; A61K 9/1688

(57) ABSTRACT

The present invention relates to a process for the preparation of spherical agglomerates of timapiprant; the invention also relates to spherical agglomerates of timapiprant obtained by the above process and pharmaceutical formulation comprising said agglomerates of timapiprant.

### FIELD OF THE INVENTION

The present invention relates to a process for the preparation of spherical agglomerates of timapiprant. The invention also relates to spherical agglomerates of timapiprant obtained by said process and pharmaceutical formulation comprising said agglomerates of timapiprant.

### BACKGROUND OF THE INVENTION

Timapiprant is a potent, selective, and orally active prostaglandin D2 receptor (CRTH2) antagonist, that retains activity in human whole blood and inhibits mast cell-dependent activation of both human Th2 lymphocytes and eosinophils.

PGD2 is an eicosanoid, a class of chemical mediator synthesised by cells in response to local tissue damage, normal stimuli or hormonal stimuli or via cellular activation pathways. Eicosanoids bind to specific cell surface receptors on a wide variety of tissues throughout the body and mediate various effects in these tissues. PGD2 is known to be produced by mast cells, macrophages and Th2 lymphocytes and has been detected in high concentrations in the airways of asthmatic patients challenged with antigen (Murray et al., (1986), N Engl. J Med 315: 800-804). Instillation of PGD2 into airways can provoke many features of the asthmatic response including bronchoconstriction (Hardy et al., (1984) N Engl. J Med 311: 209-213; Sampson et al. (1997) Thorax 52: 513-518) and eosinophil accumulation (Emery et al., (1989) J Appl. Physiol. 67: 959-962).

Clinical studies have demonstrated that the timapiprant is effective in reducing airway inflammation and improving lung function and quality of life in patients with atopic eosinophilic asthma.

Timapiprant was first described in EP1682121 (Atopix Therapeutics Ltd.), along with a number of indole acetic acid derivatives which are inhibitors of PGD2 at the CRTH2 receptor and which are therefore useful in the treatment or prevention of diseases and conditions such as allergic asthma, perennial allergic rhinitis, seasonal allergic rhinitis, atopic dermatitis, contact hypersensitivity (including contact dermatitis), conjunctivitis, especially allergic conjunctivitis, eosinophilic bronchitis, food allergies, eosinophilic gastroenteritis, inflammatory bowel disease, ulcerative colitis and Crohn's disease, mastocytosis and' also other PGD2-mediated diseases, for example autoimmune diseases such as hyper IgE syndrome and systemic lupus erythematus, psoriasis, acne, multiple sclerosis, allograft rejection, reperfusion injury, chronic obstructive pulmonary disease, as well as, in some cases, rheumatoid arthritis, psoriatic arthritis and osteoarthritis and neurodegenerative diseases such as Alzheimer's disease, Parkinson's disease, stroke and amyoptrophic lateral sclerosis.

The process for the preparation of timapiprant and derivatives thereof is also disclosed in some literature and patents.

EP3083557 B1 (Atopix Therapeutics Ltd.) describes the route of synthesis and names the precursor C1-C6 alkyl or benzyl ester of 3-substituted (indol-1-yl)-acetic acid esters of formula I. In particular, EP3083557 describes certain reaction conditions for stage 2 of the synthesis, together with the hydrolysis of the stage 2 product to provide the 3-substituted (indol-1-yl)-acetic acid esters of formula I the and preparation of the stage 1 product.

EP2791129 B1 (Atopix Therapeutics Ltd.), describes the routes of synthesis of 5-fluoro-2-methyl-3-(quinolin-2-ylmethyl)-1H-indol-1-yl)acetic acid and its derivatives. In particular, the invention describes and exemplifies the hydrolysis of the stage 2 product in the presence of KOH in water and mentions the particular suitability of hydrochloric and formic acids for the acidification; however EP2791129 B1 does not disclose the steps leading to isolation of the 5-fluoro-2-methyl-3-(quinolin-2-ylmethyl)-1H-indol-1-yl)acetic acid. It is further described and exemplified the reagents used currently for stage 2 of the synthesis.

WO2006/092579 (Oxagen Ltd.,), describes a microcrystalline form of the timapiprant and a process for preparing said form which does not involve a milling process. In particular, the microcrystalline form is prepared by recrystallisation of 5-fluoro-2-methyl-3-quinolin-2-yl-methyl-indol-1-yl)-acetic acid from DMSO/water followed by partial dissolution in aqueous potassium carbonate, the weak base, and then acidification using citric acid, the weak acid.

Timapiprant is a poorly soluble drug substance, the process described in WO2006/092579 is capable of providing microcrystalline form of timapiprant thus enhancing the solubility and bioavailability properties.

The micrometric properties such as shape and size are essential for the formulation of solid dose unit of poorly soluble ingredients; the particle size is in fact commonly recognized as an issue for such ingredients due to its impact on dissolution properties and solubility.

In this direction, many technologies are available nowadays capable to enhance solubility and bioavailability of a drug substance. Wet milling procedure, for instance, can provide directly small particles in suspension which are typically collected by filtration. However wet milling needs additional dedicated equipment, i.e. the high energy mixer, and the stability of the active pharmaceutical ingredient (API) upon high energy milling in solution is unknown and may be different from the stability of the dry API.

Sonocrystallization can be used to produce small crystals and can be used in conjunction with continuous crystallization, but it is usually limited to small scale. In fact, it is known to present scale-up issues, being a high energy technique difficult to apply to large reactors, often showing poor reproducibility.

Spray drying of an active ingredient solution is still an alternative technique which can give materials having improved characteristics for formulation and control over particle size distribution (PSD). However, the product obtained is normally in amorphous form and non-crystalline. Furthermore, the spray drying technology requires dedicated equipment and the API should have thermal stability to high temperature.

In general term, it has to be noted that beside the advantages that the above technology can provide, said technologies have nevertheless a common main issue which is due to the fact that reducing the particle size of active ingredients, can lead to several criticalities e.g. in terms of industrial processability, especially for the manufacturing process of pharmaceutical formulation.

For example, the obtainment of timapiprant with a very fine particle size, according to WO2006/092579, implicates major disadvantages during the manufacturing process of drug product.

Particularly in this respect, the step of isolation of timapiprant by filtration is very slow with the high risk of product passing through the filter cloth due to the very fine particle size. Last, but not least, timapiprant with fine particle size has a poor flowability and it is not a suitable form for the manufacturing of a pharmaceutical formulation in solid unit. It is in fact known from the art that the flow of powder during manufacturing can have an important impact on the quality of the product, e.g. in terms of its weight and content uniformity.

Hence, there is a need in the art to find improved, efficient process for preparing a solid form of timapiprant with good filtration properties, directly suitable for dry formulation and at the same having the ability to show a convenient and/or enhanced bioavailability.

### SUMMARY OF THE INVENTION

In a first aspect, the present invention is directed to a pharmaceutical composition in a solid form comprising spherical agglomerates of timapiprant, wherein said spherical agglomerates of timapiprant are obtained by a process comprising the steps of:

a) preparing an aqueous suspension of 5-fluoro-2-methyl-3-(quinolin-2-ylmethyl)-1H-indol-1-yl)acetic acid of formula I;

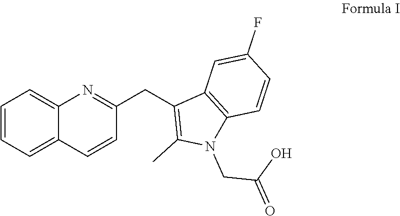 [SMILES: Cc1c(Cc2ccc3ccccc3n2)c2cc(F)ccc2n1CC(=O)O]

b) agglomerating the particles of 5-fluoro-2-methyl-3-(quinolin-2-ylmethyl)-1H-indol-1-yl)acetic acid obtained in step a) by addition of an agglomerating agent;

c) isolating the agglomerates obtained in step b); and optionally

d) washing and drying the isolated agglomerates.

In a second aspect, the present invention is directed to a process for the preparation of the pharmaceutical composition, comprising the steps of:

a) preparing an aqueous suspension of 5-fluoro-2-methyl-3-(quinolin-2-ylmethyl)-1H-indol-1-yl)acetic acid of formula I;

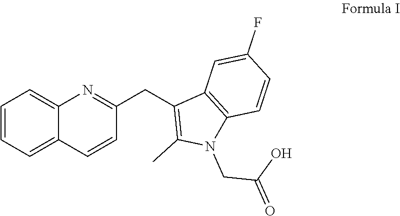 [SMILES: Cc1c(Cc2ccc3ccccc3n2)c2cc(F)ccc2n1CC(=O)O]

b) agglomerating the particles of 5-fluoro-2-methyl-3-(quinolin-2-ylmethyl)-1H-indol-1-yl)acetic acid obtained in step a) by addition of an agglomerating agent;

c) isolating the agglomerates obtained in step b);

d) washing and drying the isolated agglomerates;

e) mixing the isolated agglomerates obtained in step d) with pharmaceutical acceptable excipients;

f) compressing the mixture obtained in step e) to obtain tablets.

In a third aspect, the present invention is directed to a pharmaceutical composition in a solid form comprising spherical agglomerates of timapiprant obtained as above described for use in the treatment of respiratory diseases.

### BRIEF DESCRIPTION OF THE FIGURES

FIG. 1: SEM image of the secondary particles of spherical agglomerates of Timapiprant.

FIG. 2: SEM image of secondary and primary particles of spherical agglomerates of Timapiprant obtained according to Example 3.

FIG. 3: SEM image of secondary and primary particles of spherical agglomerates of Timapiprant obtained according to Example 5.

FIG. 4: PSD curve by laser diffraction of primary particles of Timapiprant after break of agglomerates obtained according to Example 3.

FIG. 5: PSD curve by laser diffraction of primary particles of Timapiprant after break of agglomerates obtained according to Example 5.

FIG. 6: SEM image of spherical agglomerates of Timapiprant obtained according to Example 5, in the excipients mixture pre-compression.

FIG. 7: SEM image of spherical agglomerates of Timapiprant obtained according to Example 5, in the excipients mixture pre-compression.

FIG. 8: SEM image of spherical agglomerates of Timapiprant obtained according to Example 5, in the excipients mixture pre-compression.

FIG. 9: Raman mapping of timapiprant spherical agglomerates performed on the Tablets A of Example 8.

FIG. 10: Raman mapping of timapiprant spherical agglomerates performed on the Tablets A of Example 8.

FIG. 11: Comparison of dissolution profiles of Tablets A and Tablets B.

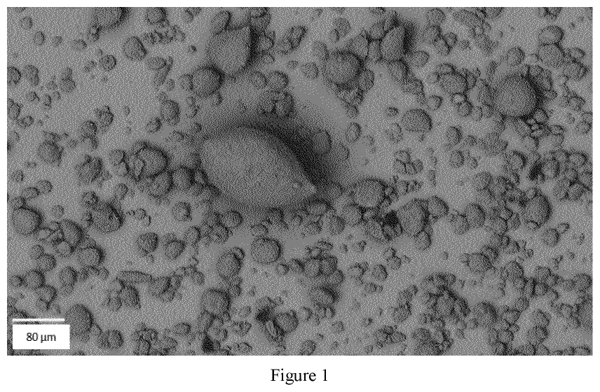

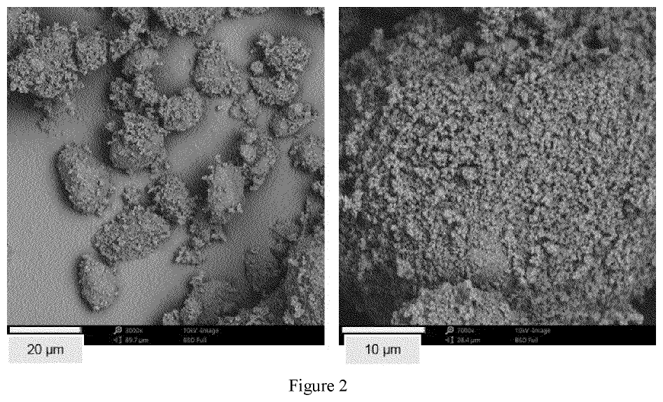

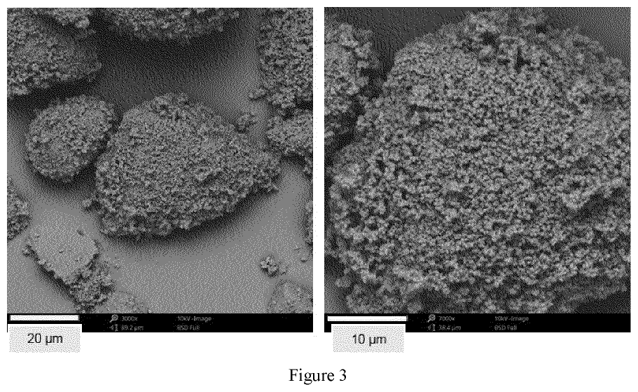

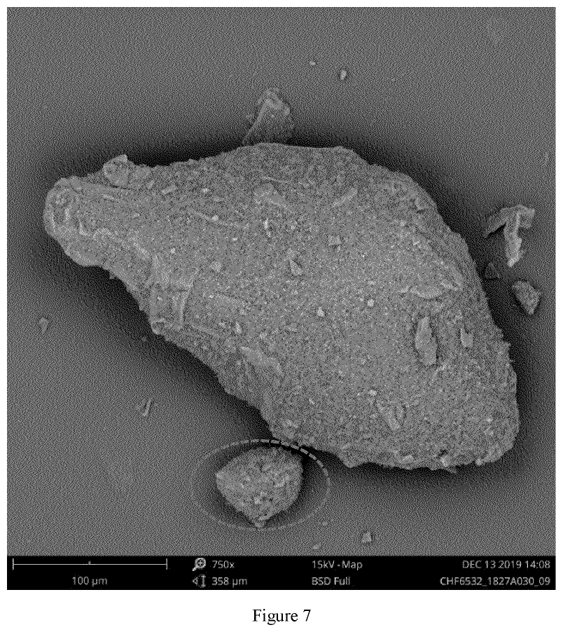

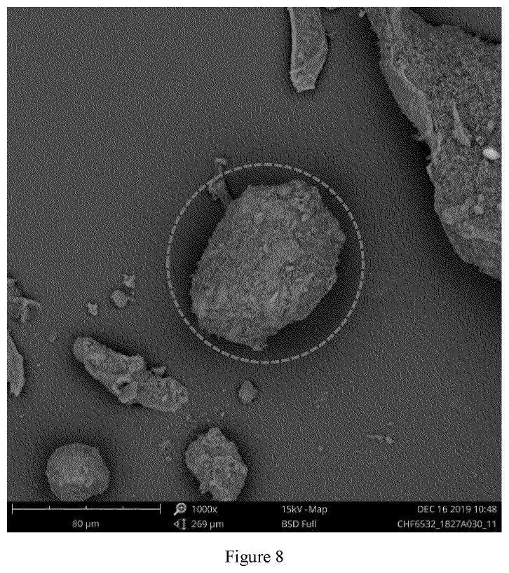

The scanning electron microscopy (SEM) images of FIGS. 1, 2, 3, 6, 7 and 8 were measured by means of a Phenom XL using a backscattered electron detector with intensity at 15 kV set in image mode.

The Raman analyses were run on a Keiser Optical Systems RXN1 MicroRaman with Leica Microscope and digital camera.

### DETAILED DESCRIPTION OF THE INVENTION

Definitions

In each aspect of the present invention, the term “timapiprant” encompasses the compound of Formula I, chemically designated as 5-fluoro-2-methyl-3-(quinolin-2-ylmethyl)-1H-indol-1-yl)acetic acid, as well as, pharmaceutically acceptable salts thereof

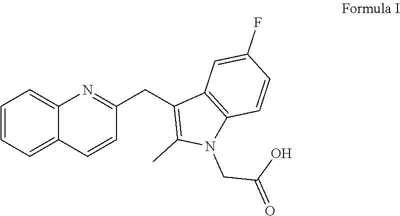 [SMILES: Cc1c(Cc2ccc3ccccc3n2)c2cc(F)ccc2n1CC(=O)O]

Unless otherwise specified, the terms “active drug”, “active ingredient”, “active” and “active substance”, “active compound” and “therapeutic agent” and “API” are used as synonymous.

The term “micron” indicated with “μm” is used as synonymous of “micrometers”, i.e. 10⁻⁶ m.

In general terms, the size of the particles can be quantified by measuring a characteristic equivalent sphere diameter, known as volume diameter, by laser diffraction, according to European Pharmacopeia 8.0; 2.9.31; “Particle size analysis by laser light diffraction”.

The particle size can also be quantified by measuring the mass diameter by means of suitable instruments and techniques known to the skilled person, such as sieving.

The volume diameter (VD) is related to the mass diameter (MD) by the density of the particles (assuming the size being independent of the density of the particles).

In the present application, the particle size may be expressed in terms of volume diameter. In particular, the particle size distribution is expressed in terms of: i) the volume median diameter (VMD) which corresponds to the diameter of 50 percent by weight of the particles, i.e. d(v0.5), and ii) the volume diameter (VD) in micron of 10% and 90% of the particles, respectively, i.e. d(v0.1) and d(v0.9), according to European Pharmacopeia 8.0; 2.9.31; “Particle size analysis by laser light diffraction”.

The d(v0.1), d(v0.5) and d(v0.9) values in μm refers to the particle size distribution diameters below which are found 10% (D10), 50% (D50) and 90% (D90) respectively of the population, according to European Pharmacopeia 8.0; 2.9.31; “Particle size analysis by laser light diffraction”.

Alternatively, the particle size interval may be expressed in terms of mass diameter. In particular, the particle size distribution is expressed in terms of: i) the mass median diameter (MMD) which corresponds to the diameter of 50 percent by weight of the particles, e.g. d(0.5), and ii) the mass diameter (MD) in micron of 10% and 90% of the particles, respectively, e.g. d(0.1) and d(0.9).

With the term “fine particles” or “small particle size” it is meant particles having a median volume diameter smaller than or equal to 3 microns.

With the term “primary particle”, it is meant the smallest subdivision of the solid material, also called “fundamental” particles, which cannot be separated into smaller particles and having a primary particle size smaller than or equal to 3 μm.

With the “primary particle size” it is meant the size of primary particles.

With the term “secondary particle” it is meant assembly of primary particles tightly bound together by strong cohesion forces resulting in characteristic and unique agglomerates, and having a secondary particle size greater than 3 μm.

With the term “secondary particle size” it is meant the size of secondary particles.

With the “spherical agglomerates particle size” or when the “particle size” refers to the spherical agglomerates, it is assumed to be the secondary particle size.

The acronym “PSD” refers to particle size distribution.

The term “good flow properties” refers to a powder or dry formulation that can be easily handled during the manufacturing process and is able of ensuring an accurate and reproducible delivering of the therapeutically effective dose.

The term “free-flowing solid form” or “free-flowing form” relates to a solid form wherein the particles do not stick together or cohesive when cling to one another, as further described in reference “On powder Flowability”, J. Prescott Pharmaceutical Technology, 2000.

The term of “good filtration properties” refers to material that can be conveniently and effectively filtered without affecting the duration or the yield of the overall process of manufacturing.

The term “disagglomeration” refers to the breakage of the spherical agglomerates of timapiprant.

The term “agglomerating agent” refers to a substance, typically a liquid, which is able to generate solid agglomerates having good filtration properties as above defined, when generally added to a proper suspension.

The term “suspension” refers to a heterogeneous mixture in which particles are suspended or partially suspended throughout the bulk of the solvent, contrary to a “solution” that refers to a homogeneous mixture, wherein a substance (solute) is dissolved in another substance known as a solvent. The term “aqueous solution” refers to a solution when the solvent is water, substantially free of particles, solids or undissolved material.

The term “Specific surface area” or “SSA” refers to a property of solids defined as the total surface area of a material per unit of mass with units of m²/kg or m²/g.

The acronym “FBRM” refers to focused beam reflectance measurement that is an increasingly popular particle growth analysis technique.

The present invention provides a process for the manufacturing of spherical agglomerates of timapiprant, spherical agglomerates of timapiprant obtained by the process on the invention and pharmaceutical compositions comprising said agglomerates.

We have now, surprisingly, found an efficient process via spherical agglomeration to prepare a form of timapiprant having good filtration properties, in a free-flowing solid form ready for the formulation and having an enhanced bioavailability.

The process of the present invention provides a series of advantages, such as making feasible and convenient the filtration of timapiprant through a convenient agglomeration into larger and secondary particles, the spherical agglomerates, substantially avoiding the use of dedicated or specialized equipment and the thermal stress. Even more, the process of the present invention is applicable to industrial scale ensuring high yields.

The spherical agglomerates obtained by the process of the invention have particle size, also defined as secondary particle size, greater than 3 μm.

Said agglomerates can be filtered using standard equipment, the yield of the filtration step is significantly improved over the prior art, with a yield even greater than about 90%.

As a further advantage, the isolated agglomerates obtained by the process of the invention present improved mechanical and flow properties, facilitating operation of the formulation steps, i.e. dry compression. The isolated agglomerates are a free-flowing solid form which can be directly used for the preparation of pharmaceutical formulations as below described in detail.

The spherical agglomerates of timapiprant obtained by the process of the present invention can be directly compressed to obtain tablets at industrial scale level without any additional intermediate step, i.e. granulation, that could have an impact on the manufacturing process. Typically, the granulation step increases the manufacturing cost and the timing to produce an industrial batch of tablets.

As can be appreciated in Example 8, the spherical agglomerates of timapiprant can be easily worked to obtain firstly a mixture together with the excipients; said mixture is then directly compressed to obtain the tablets, according to the present invention. The Example 6 also shows that, as a comparison, the direct compression cannot be applied to the mixture of micronized timapiprant together with excipients. In this latter case in fact the mixture showed very poor flow properties and strong adhesion to the walls of the powder funnel, thus compromising a possible use at industrial level.

Advantageously, the spherical agglomerates of timapiprant obtained by the process of the invention, can break up either during the manufacturing process of formulation, for example during a compression step, or even after the administration to the subjects, thus releasing particles of timapiprant having a primary particle size smaller than or equal to 3 μm. This is a further advantage of the process of the invention that beside improving the filtering process, it also may end in providing a solid form of timapiprant with a primary particle size suitable to ensure a good bioavailability when administered.

The spherical agglomerates of timapiprant can partially break up during the manufacturing process to obtain the pharmaceutical formulation, such as tablets. For example, the spherical agglomerates of timapiprant can break up during the mixing step with the excipients, or for example during the compression step when the formulation is a tablet; therefore, a part of spherical agglomerates can be identified in the mixture with the excipients pre-compression and even in the tablets.

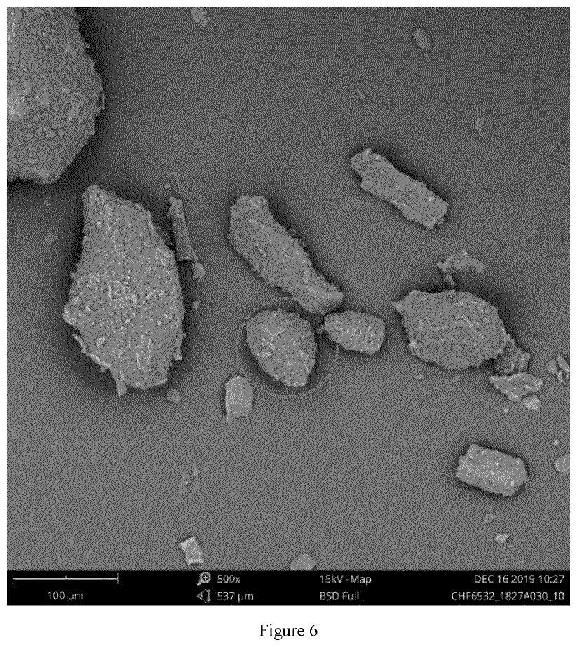

As can be appreciated in the FIGS. 6, 7 and 8 the particles circled in red can be identified as the spherical agglomerates of timapiprant that are clearly distinguishable from the other particles made of excipients. The identification of the spherical agglomerates of timapiprant is analyzed by means of energy-dispersive X-ray spectroscopy (EDS) using a Detector type Silicon Drift Detector, that allows to measure the atomic composition of the area observed by scanning electron microscopy (SEM). The signal collected over one of the spherical agglomerate of timapiprant indicates at least 15% of nitrogen atoms present, this value is an evidence that the agglomerate is composed by the timapiprant.

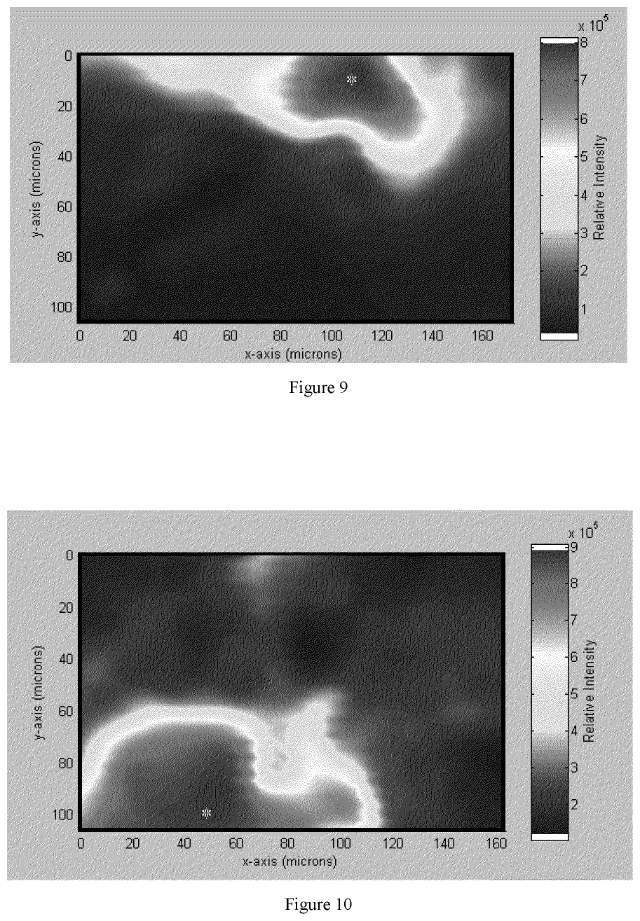

The FIGS. 9 and 10 show the Raman Map of tablets comprising the spherical agglomerates of timapiprant wherein the areas in red indicate the presence of agglomerates of timapiprant with respect to the matrix.

As above described, the particle size is commonly recognized as an issue for poorly soluble ingredients, such as timapiprant, due to its impact on dissolution properties. Therefore, it is required to have a fine particle size in order to maximize the surface area which, in turn, maximizes the oral absorption by the body from the gastrointestinal tract.

Thus, in this respect, the primary particle size of timapiprant after breaking up the agglomerates obtained by the process of the invention, preferably shows the following values for the PSD parameters: D10 below 0.5 μm and/or D50 below 1 μm.

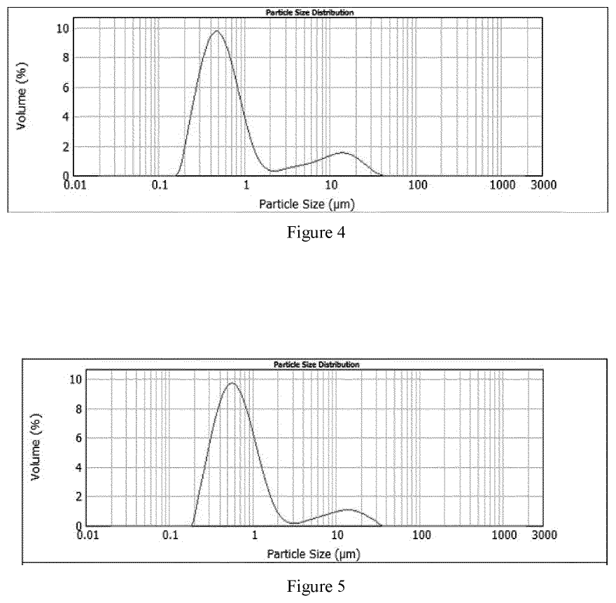

The primary particle size distribution curves are shown in FIGS. 4 and 5. As it can be seen from said figures, the D50 values are under 1 μm whereas the D10 values are under 0.5 μm. Beyond the primary particle size, the spherical agglomerates of timapiprant are further characterized by a specific surface area (SSA) greater than the specific surface area of micronized timapiprant.

As can be appreciated in Table 9, the specific surface area of micronized timapiprant determined by BET nitrogen adsorption as reported in Example 10, is less than 8.5 m²/g, significantly lower compared to the specific surface area of spherical agglomerates of timapiprant that reaches value up to 15.5 m²/g.

Therefore, the process of the invention provides a form of timapiprant with improved filtration, mechanical and flow properties which facilitate the overall process for the preparation of a drug product, providing at the same time a particle size suitable to ensure a good absorption when administered. This peculiar aspect is also confirmed by the dissolution profile of tablets comprising the spherical agglomerates of timapiprant obtained according to the process of the present invention, comparable to the dissolution profile of micronized timapiprant.

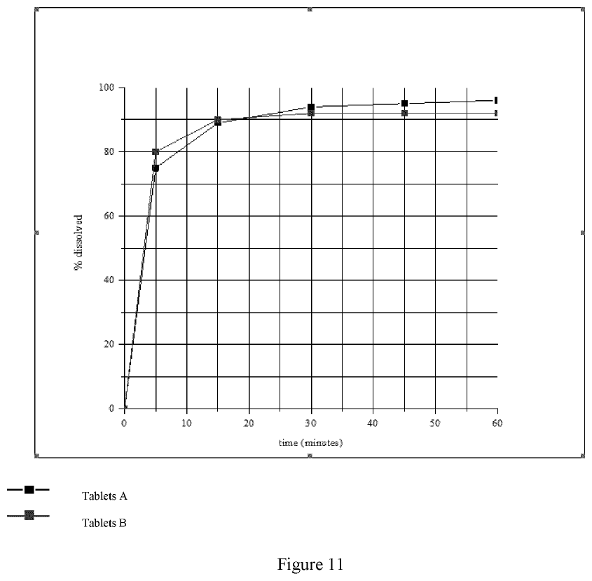

The Table 8 and FIG. 11 show that both Tablets A comprising spherical agglomerates and Tablets B comprising the micronized form, when measured using a paddle Type-II dissolution apparatus according to USP <711>, in 900 mL of 50 mM monosodium phosphate buffer, at paddle speed of 75 RPM and at a temperature of 37° C., release at least 75% of the timapiprant after 5 minutes, and more than 90% after 60 minutes. These dissolution profiles are in compliance with an immediate release formulation for a poorly soluble active ingredient.

As above mentioned, one aspect of the present invention refers to a process for the preparation of spherical agglomerates of timapiprant, said process comprising the steps of:

a) preparing an aqueous suspension of 5-fluoro-2-methyl-3-(quinolin-2-ylmethyl)-1H-indol-1-yl)acetic acid of formula (I) as above indicated;

b) agglomerating the particles of 5-fluoro-2-methyl-3-(quinolin-2-ylmethyl)-1H-indol-1-yl)acetic acid obtained in step a) by addition of an agglomerating agent;

c) isolating the agglomerates obtained in step b); and optionally

d) washing and drying the isolated agglomerates obtained.

In one embodiment the aqueous suspension of step a) can be prepared by adding an acid to a salt of 5-fluoro-2-methyl-3-(quinolin-2-ylmethyl)-1H-indol-1-yl)acetic acid to form the suspension of 5-fluoro-2-methyl-3-(quinolin-2-ylmethyl)-1H-indol-1-yl)acetic acid. The salt is an alkaline salt selected from sodium, lithium and potassium, preferably a, or an ammonium salt. Preferably the salt is potassium salt.

Thus in one preferred embodiment, the process for the preparation of spherical agglomerates of timapiprant of the invention, comprises the steps of:

a) preparing an aqueous suspension of 5-fluoro-2-methyl-3-(quinolin-2-ylmethyl)-1H-indol-1-yl)acetic acid of formula (I) as above indicated wherein said preparation comprises the step of:

ii) adding an acid to an aqueous solution of a salt of 5-fluoro-2-methyl-3-(quinolin-2-ylmethyl)-1H-indol-1-yl)acetic acid;

b) agglomerating the particles of 5-fluoro-2-methyl-3-(quinolin-2-ylmethyl)-1H-indol-1-yl)acetic acid obtained in step a) by addition of an agglomerating agent;

c) isolating the agglomerates obtained in step b); and optionally

d) washing and drying the isolated agglomerates obtained.

In one embodiment, the solution of step ii) can be prepared by dissolution of 5-fluoro-2-methyl-3-quinolin-2-yl-methyl-indol-1-yl)acetic acid in any suitable base such as, but not limited to, lithium, sodium, potassium or ammonium hydroxide to form an alkaline or ammonium salt in aqueous solution. Preferably the base is potassium hydroxide to obtain a potassium salt. The resulting aqueous solution of the salt of 5-fluoro-2-methyl-3-(quinolin-2-ylmethyl)-1H-indol-1-yl)acetic acid, preferably potassium salt, is then acidified with an acid in order to obtain the desired aqueous suspension of 5-fluoro-2-methyl-3-(quinolin-2-ylmethyl)-1H-indol-1-yl)acetic acid, suitable to be used in the step a) of the present process.

In one preferred embodiment, the aqueous suspension of step a) can be directly obtained at the end of the process of synthesis of timapiprant, as described in European patent no. EP2791129 B. Therefore, the present invention also refers in one embodiment to a process for the preparation of timapiprant, wherein the compound of formula (I) in step a) is obtained by an ester-hydrolysis reaction by treatment of 5-fluoro-2-methyl-3-(quinolin-2-ylmethyl)-1H-indol-1-yl)acetic acid, ethyl ester with a base, according to the teaching of EP2791129 B. In particular, the process for the preparation of spherical agglomerates of timapiprant, comprises the steps of:

a) preparing an aqueous suspension of 5-fluoro-2-methyl-3-(quinolin-2-ylmethyl)-1H-indol-1-yl)acetic acid of formula (I) wherein said preparation comprises the steps of:
i) adding a base to 5-fluoro-2-methyl-3-(quinolin-2-ylmethyl)-1H indol-1-yl)acetic acid, ethyl ester in the presence of water;
ii) adding an acid to the aqueous solution of a salt of 5-fluoro-2-methyl-3-(quinolin-2-ylmethyl)-1H-indol-1-yl)acetic acid obtained in step i);

b) agglomerating the particles of 5-fluoro-2-methyl-3-(quinolin-2-ylmethyl)-1H-indol-1-yl)acetic acid obtained in step a) by addition of an agglomerating agent;

c) isolating the agglomerates obtained in step b); and optionally

d) washing and drying the isolated agglomerates obtained.

In one embodiment the base added to 5-fluoro-2-methyl-3-quinolin-2-yl-methyl-indol-1-yl)acetic acid ethyl ester of step i) is selected from: lithium, sodium, potassium and ammonium hydroxide, preferably potassium hydroxide. The amount of base added in step i) is comprised between 1 and 5 equivalents with respect to 5-fluoro-2-methyl-3-quinolin-2-yl-methyl-indol-1-yl)acetic acid ethyl ester, preferably between 1 and 3.

After the addition of the selected base, the mixture of 5-fluoro-2-methyl-3-quinolin-2-yl-methyl-indol-1-yl)acetic acid, ethyl ester in aqueous potassium hydroxide and water is stirred. The reaction is carried out at temperature between 40° C. and 80° C., preferably between 50° C. and 70° C., more preferably between 60° C. and 65° C. The mixture is generally held at this temperature until completion of the hydrolysis.

In one embodiment the mixture may be optionally cooled at 20° C. and then heated again at temperature comprised between 60° C. and 65° C. This cooling step is useful e.g. to increase the yield of the reaction.

A polish filtration might be applied at this stage in order to remove foreign particles potentially present in the solution.

The resulting aqueous solution of the salt of 5-fluoro-2-methyl-3-(quinolin-2-ylmethyl)-1H-indol-1-yl)acetic acid is then acidified with an acid according to step ii).

The addition of the acid leads to the formation of a suspension of 5-fluoro-2-methyl-3-(quinolin-2-ylmethyl)-1H-indol-1-yl)acetic acid; said suspension of 5-fluoro-2-methyl-3-(quinolin-2-ylmethyl)-1H-indol-1-yl)acetic acid is characterized by having a fine particle size particularly suitable to be agglomerated via spherical agglomeration according to the process of the present invention.

The obtainment of the aqueous suspension of step a) directly at the end of the process of synthesis of timapiprant is even more advantageous, since the process can be carried out as continually without interruption, thus increasing the overall yield of the process and reducing timing and costs.

In one further embodiment, the aqueous suspension of step a), can be obtained re-processing agglomerates of 5-fluoro-2-methyl-3-quinolin-2-yl-methyl-indol-1-yl)acetic acid obtained according to the above process, by their dissolution in any suitable base such as, but not limited to, lithium, sodium, potassium or ammonium hydroxide, preferably potassium hydroxide, heating the solution at a temperature comprised between 40° C. and 80° C. to form the salt in aqueous solution. Preferably, the temperature is comprised between 50° C. and 70° C., more preferably between 60° C. and 65° C.

When the 5-fluoro-2-methyl-3-quinolin-2-yl-methyl-indol-1-yl)acetic acid is re-processed, ethanol is optionally added to the aqueous potassium hydroxide before or after heating. Also in this case, the resulting aqueous solution of the salt of 5-fluoro-2-methyl-3-(quinolin-2-ylmethyl)-1H-indol-1-yl)acetic acid is acidified with an acid in order to obtain an aqueous suspension of 5-fluoro-2-methyl-3-(quinolin-2-ylmethyl)-1H-indol-1-yl)acetic.

In one embodiment, the addition of acid to the aqueous solution of the salt of 5-fluoro-2-methyl-3-(quinolin-2-ylmethyl)-1H-indol-1-yl)acetic acid of step ii) is performed at suitable temperature to obtain a suspension of the 5-fluoro-2-methyl-3-(quinolin-2-ylmethyl)-1H-indol-1-yl)acetic acid in a form having a fine particle size.

The temperature is comprised between about 40° C. and about 80° C.; preferably between about 50° C. and about 70° C., more preferably between about 60° C. and about 65° C.

In a further embodiment the amount of acid added of step ii) is comprised between 1 and 15 mol/mol in respect of 5-fluoro-2-methyl-3-quinolin-2-yl-methyl-indol-1-yl)acetic acid or its corresponding ethyl ester, preferably from 1 to 10, more preferably from 1 to 7, preferably from 1 to 5.

In a further embodiment, suitable acids for use in the method of the invention are organic or inorganic acids. Preferably the acid is an organic acid selected from formic, acetic, propionic, succinic, malic, maleic, fumaric, citric acid. More preferably the acid is formic acid.

The obtained aqueous suspension of step a) is held at a temperature comprised between about 40° C. and 80° C., preferably between 50° C. and 70° C., more preferably between 60° C. and 65° C., in order to obtain a more flowing, stirrable suspension. The suspension is held at the selected temperature for a period between 20 minutes and up to 2 hours. Preferably, the period is between 30 minutes and 1 hour.

The aqueous suspension of step a) is then optionally cooled to a temperature comprised between 25° C. and 50° C., depending on the temperature conditions of step a), because the following step b) of the process typically requires a lower temperature respect to step a).

The fine particles of 5-fluoro-2-methyl-3-(quinolin-2-ylmethyl)-1H-indol-1-yl)acetic acid in aqueous suspension obtained in step a) are agglomerated by the addition of an agglomerating agent at a temperature comprised between 25° C. and 50° C., more preferably between 30° C. and 40° C., and holding at this temperature with appropriate stirring, suitable to maintain flowability of the mixture, until the agglomeration is considered to have proceeded to a sufficient extent, based e.g. on in-line analysis using FBRM.

In the present invention, the addition of an agglomerating agent triggers the agglomeration following the mechanism as described in the review “Particle design via spherical agglomeration: A critical review of controlling parameters, rate processes and modelling.” Kate Pitt et al.; Powder Technology 326 (2018) 327-343.

In one embodiment, suitable agglomerating agent for use in the process of the present invention is selected from: methyl isobutyl ketone, isopropyl acetate, cyclopentyl methyl ether (CPME) and toluene. In a preferred embodiment, the agglomerating agent is toluene.

In a further embodiment the amount of agglomerating agent is comprised between 0.40 to 0.80 kg/kg in respect of 5-fluoro-2-methyl-3-(quinolin-2-ylmethyl)-1H-indol-1-yl)acetic acid or its corresponding ethyl ester, more preferably between 0.55 and 0.65 kg/kg.

The growth of the particles during the agglomeration step b) can be monitored using e.g. in-line FBRM monitoring. The equipment that can be used is Mettler-Toledo Particle track™ G600Ex with 25 mm diameter probe. The chord length distribution tracks how particle size and count change during the process. A ‘no weight’ method is used, for which a target for the 50 μm population of <5.0% has been set and agglomeration is considered complete when this criterion is achieved.

According to one embodiment of the present invention, the agglomeration occurs in a period comprised between 5 hours and 48 hours, more preferably between 12 and 24 hours.

In one embodiment of the invention, the aqueous suspension of 5-fluoro-2-methyl-3-(quinolin-2-ylmethyl)-1H-indol-1-yl)acetic acid of step a) is heated and/or held at a temperature comprised between 40° C. and 80° C. even when comprehensive of steps i) and ii) as afore described in details, and the agglomeration of step b) is carried out at a temperature comprised between 25° C. and 50° C.

In one embodiment of the invention, the aqueous suspension of 5-fluoro-2-methyl-3-(quinolin-2-ylmethyl)-1H-indol-1-yl)acetic acid of step a) is heated and/or held at temperature comprised between 60° C. and 65° C. even when comprehensive of steps i) and ii) as afore described in details, and the agglomeration of step b) is carried out at temperature comprised between 25° C. and 50° C.

In a further preferred embodiment, the aqueous suspension of 5-fluoro-2-methyl-3-(quinolin-2-ylmethyl)-1H-indol-1-yl)acetic acid of step a) is heated and/or held at temperature comprised between 60° C. and 65° C., even when comprehensive of steps i) and ii) as afore described in details, and the agglomeration of step b) is carried out at temperature comprised between 30° C. and 40° C.

The resulting spherical agglomerates of timapiprant obtained in step b) are then isolated by filtration, typically using standard equipments.

The isolated agglomerates of step c) can be optionally washed and dried. Preferably, the isolated agglomerates are washed with water and then dried under vacuum.

The yield of the isolation step calculated after washing and drying of the spherical agglomerates of timapiprant obtained by the process of the invention is about 90% on average as shown in the Examples 1 to 5.

In one embodiment, the dried product is optionally passed through a mechanical sieve and subsequently homogenized if necessary. This latter step is particularly advantageous when large-scale operation is considered. In this respect, it has to be highlighted that beside the many advantages of the invention as herein described in details, the present process can be also used at industrial level, when high amounts of final product timapiprant are required, also in light of its versatility and reproducibility.

In one aspect, the present invention provides spherical agglomerates of timapiprant obtained, or obtainable, by the process of the present invention, as above described.

In one embodiment, the particle size of the spherical agglomerates of timapiprant obtained by the process of the invention is greater than 3μm. Preferably, the spherical agglomerates of timapiprant have a particle size comprised between 3 and 10 μm.

In a further embodiment, the particle size of the spherical agglomerates of timapiprant obtained by the process of the invention is greater than 10 μm.

Preferably the spherical agglomerates of timapiprant have a particle size comprised between 10 and 300 μm.

Preferably the spherical agglomerates of timapiprant have a particle size comprised between 10 and 200 μm.

Preferably the spherical agglomerates of timapiprant have a particle size comprised between 10 and 50 μm.

Preferably the spherical agglomerates of timapiprant have a particle size comprised between 3 and 50 μm.

The spherical agglomerates obtained by the process of the present invention, are shown in FIGS. 1, 2 and 3.

In one embodiment, the spherical agglomerates of timapiprant obtained by the process of the present invention have a specific surface area determined by BET nitrogen adsorption greater than 9 m²/g. Preferably the spherical agglomerates of timapiprant have a specific surface area comprised between 9 and 40 m²/g. Preferably the spherical agglomerates of timapiprant have a specific surface area comprised between 9 and 30 m²/g. More preferably the spherical agglomerates of timapiprant have a specific surface area comprised between 9 and 20 m²/g; even more preferably the spherical agglomerates of timapiprant have a specific surface area comprised between 10 and 18 m²/g.

The spherical agglomerates of timapiprant obtained by the process of the present invention have very good filtration properties and they are in a free-flowing suitable form ready to be used for the preparation of pharmaceutical formulation.

In one preferred embodiment, the agglomerates of timapiprant can be used directly for the preparation of pharmaceutical formulation in a solid form.

In one aspect, the invention refers to a pharmaceutical composition in a solid form comprising the spherical agglomerates of timapiprant obtained by the process of the present invention.

In a further preferred embodiment, the pharmaceutical composition in a solid form is selected from the group comprising tablets, capsules, pills, sachets and granules.

In a further preferred embodiment, the pharmaceutical composition in a solid form is a tablet.

In one preferred embodiment, the pharmaceutical composition in a solid form comprises spherical agglomerates of timapiprant obtained by the process of the present invention characterized by having a specific surface area determined by BET nitrogen adsorption greater than 9 m²/g. Preferably the spherical agglomerates of timapiprant have a specific surface area comprised between 9 and 40 m²/g, more preferably between 9 and 30 m²/g, more preferably between 9 and 20 m²/g, even more preferably between 10 and 18 m²/g.

In one preferred embodiment, the pharmaceutical composition is a tablet comprising spherical agglomerates of timapiprant obtained by the process of the present invention characterized by having a specific surface area determined by BET nitrogen adsorption greater than 9 m²/g. Preferably the spherical agglomerates of timapiprant have a specific surface area comprised between 9 and 40 m²/g, more preferably between 9 and 30 m²/g, more preferably between 9 and 20 m²/g, even more preferably between 10 and 18 m²/g.

In one preferred embodiment, the pharmaceutical composition is a tablet comprising spherical agglomerates of timapiprant obtained by the process of the present invention, said agglomerates characterized by having a particle size greater than 10 μm and a specific surface area determined by BET nitrogen adsorption greater than 9 m²/g.

In a further preferred embodiment, the pharmaceutical composition is a tablet comprising spherical agglomerates of timapiprant obtained by the process of the present invention, said agglomerates characterized by having a particle size between 10 and 300 μm, preferably between 10 and 200 μm, more preferably between 10 and 50 μm and a specific surface area determined by BET nitrogen adsorption comprised between 9 and 40 m²/g, preferably between 9 and 30 m²/g, more preferably between 9 and 20 m²/g, even more preferably between 10 and 18 m²/g.

In one embodiment, the pharmaceutical composition in forma of tablet releases at least 75% of timapiprant at 5 minutes and 90% of timapiprant at 60 minutes when tested in a Paddle Type-II dissolution apparatus according to UPS <711>, in 900 mL of 50 mM monosodium phosphate buffer, at paddle speed of 75 RPM and at a temperature of 37° C.

The formulations of the present invention may be prepared by any methods well known in the art of pharmacy.

The route of administration may depend e.g. upon the condition to be treated but preferred compositions are formulated for oral, nasal, bronchial or topical administration.

The composition may be prepared by bringing into association the above defined

active agent with a proper carrier. In general, the formulations are prepared by uniformly and intimately bringing into association the active agent with liquid carriers or finely divided solid carriers or both, and then if necessary, shaping the product according e.g. to known methodologies.

In one further aspect, the invention provides a process for preparing a pharmaceutical composition comprising the steps a) to c) and optionally d) as above described and a further step of mixing the obtained spherical agglomerates with a pharmaceutically or acceptable excipient and/or carrier.

In one embodiment, the invention provides a process for preparing a pharmaceutical composition comprising the steps a) to c) and optionally d) as above described and a further step of mixing the obtained spherical agglomerates with a pharmaceutically acceptable excipients, wherein the formulation is for oral administration.

In one preferred embodiment, the invention provides a process for preparing a pharmaceutical composition in form of a tablet comprising the steps a) to d) as above described, a step e) of mixing the obtained spherical agglomerates with pharmaceutically acceptable excipients, and a further step g) of direct compression of the mixture obtained in step e) to obtain tablets.

In one preferred embodiment, the process for preparing a pharmaceutical composition in form of a tablet, may optionally comprise an additional granulation step of the mixture obtained in step e) before the compression of the step g).

In one preferred embedment, the process for preparing a pharmaceutical composition in form of a tablet according to the invention, may optionally comprise an additional granulation step of spherical agglomerates of timapiprant before the mixing with the excipients, e.g. before the above indicated step e).

In one preferred embodiment, the granulation is a wet or dry granulation.

Formulations for oral administration in the present invention may be presented as: discrete units such as capsules, sachets or tablets each containing a predetermined amount of the active agent.

In one preferred embodiment the pharmaceutical formulation comprising spherical agglomerates of timapiprant is a tablet.

In one further preferred embodiment, the spherical agglomerates comprised in the tablets release timapiprant with a particle size less or equal than 3 μm.

For compositions for oral administration for example, but not limited to tablets, capsules, pills, sachets and granules, the term “acceptable carrier” includes vehicles such as common excipients e.g. diluents, binders, lubricants, disintegrating agents, surfactants, sweetening agents, flavouring agents, coloring agents, coating agents and wetting agent.

Examples of pharmaceutically acceptable diluents include, but not limited to, magnesium stearate, lactose, lactose monohydrate, microciystalline cellulose, starch, pre-gelatinized starch, calcium phosphate, calcium sulfate, calcium carbonate, mannitol, sorbitol, xylitol, sucrose, maltose, fructose and dextrose.

Examples of pharmaceutically acceptable binders include, but not limited to, starches, natural sugars, corn sweeteners, natural and synthetic gums, cellulose derivatives such a methylcellulose, ethylcellulose, sodium carboxymethylcellulose hydroxypropylmethylcellulose, gelatin, PVP, polyethylene glycol, waxes, sodium alginate, alcohols,

Examples of pharmaceutically acceptable lubricants include, but not limited to metallic stearates such as magnesium stearate, metallic lauryl sulfates, fatty acids, fatty acid esters, fatty alcohols, paraffins, hydrogenated vegetable oils, polyethylene glycols, boric acid, sodium benzoate, sodium acetate, sodium chloride and talk.

Examples of pharmaceutically acceptable disintegrating agents include, but not limited to, starches, cellulose derivatives such as croscarmellose sodium, PVP, crospovidone, clays, ion-exchange resins, alginic acid and sodium alginate.

Examples of pharmaceutically acceptable surfactants include, but not limited to sulfates, sulfonates, phosphates, carboxylates, primary-secondary-tertiary amines, quaternary ammonium compounds, fatty alcohols, sugar esters of fatty acids, glycerides of fatty acids, polyoxy ethylene glycol alkyl ethers, polisorbates, sorbitan alkyl esters, and poloxamers such as poloxamer 188, metallic lauryl sulfates such as sodium lauryl sulfate.

Examples of pharmaceutically acceptable glidants include, but not limited to magnesium, silicon dioxide such as colloidal silicon dioxide, talc, starch, titanium dioxide, and the like.

Flavouring agents such as peppermint, oil of wintergreen, cherry flavouring and the like can also be used. It may be desirable to add a colouring agent to make the dosage form readily identifiable. Tablets may also be coated by methods well known in the art.

Suitable coating materials are known in the art, and include, but are not limited to, cellulosic polymers such as hydroxypropylmethylcellulose, hydroxypropylcellulose and microcrystalline cellulose, or combinations thereof (for example, various OPADRY® coating materials).

A tablet may be made by compression or moulding, optionally with one or more accessory ingredients. Compressed tablets may be prepared by compressing in a suitable machine the spherical agglomerates, in form of powder or granules, optionally mixed with a binder, lubricant, inert diluent, preservative, surface-active or dispersing agent. Moulded tablets may be made by moulding in a suitable machine a mixture of the spherical agglomerates moistened with an inert liquid diluent. The tablets may optionally be coated or scored and may be formulated so as to provide slow or controlled release of the active agent.

Other formulations suitable for oral administration include lozenges comprising the active agent in a flavoured base, usually sucrose and acacia or tragacanth; pastilles comprising the active agent in an inert base such as gelatin and glycerin, or sucrose and acacia; and mouthwashes comprising the spherical agglomerates in a suitable liquid carrier.

The pharmaceutical formulation in solid form may comprise an amount of timapiprant comprised between 5 mg and 100 mg per unit. Preferably the amount of timapiprant is comprised between 5 mg and 50 mg per unit, more preferably the amount is comprised between 25 and 50 mg.

Typically, the dose of the timapiprant will be about 0.01 to 100 mg/kg; so as to maintain the concentration of drug in the plasma at a concentration effective to inhibit PGD2 at the CRTH2 receptor. The precise amount of timapiprant which is therapeutically effective, and the route by which such compound is best administered, is readily determined by one of ordinary skill in the art.

There is also provided the use of spherical agglomerates of timapiprant in the preparation of a medicament for the treatment of diseases and conditions mediated by PGD2 at the CRTH2 receptor, wherein the medicament also comprises an additional active ingredient useful for the treatment of the same diseases and conditions.

It is understood that all preferred groups or embodiments of the present invention described above may be combined with each other and apply as well mutatis mutandis.

The invention will now be described in greater detail with reference to the following examples.

### EXAMPLES

### Example 1

### Method for Preparing Spherical Agglomerates of Timapiprant by Re-processing of 5-fluoro-2-methyl-3-quinolin-2-yl-methyl-indol-1-yl)acetic acid.

A stirred suspension of 5-fluoro-2-methyl-3-quinolin-2-yl-methyl-indol-1-yl)acetic acid (75 g) in a mixture of 50% w/w aqueous potassium hydroxide (48.3 g, 24.2 g active, 2 mol eq.) and purified water (675 ml) was heated to 60±5° C. and held at this temperature for 30 minutes. The resulting solution was polish filtered at 60±5° C., the equipment rinsed through with purified water (75 ml) and then ethanol (12.6 ml) was added to the combined filtrates.

A solution of 80% formic acid (37.2 g, 29.8 g active, 3 mol eq.) was added to the combined filtrates over a 3 hour period at 60±5° C. After holding the resulting suspension for 30 minutes at the same temperature, the mixture was then cooled to 30±3° C. and held overnight. Toluene (45 g) was added over 50 minutes at 30±3° C. The mixture was then held at this temperature for 22 hours during which agglomeration occurred. The solid was collected by filtration, washed with purified water (9×150 ml) and then dried under vacuum at 65-70° C. to provide (5-fluoro-2-methyl-3-quinolin-2-yl-methyl-indol-1-yl)acetic acid as a yellow solid (69.7 g, 93% yield uncorrected for assay).

### Example 2

### Method for Preparing Spherical Agglomerates of Timapiprant by Re-processing of 5-fluoro-2-methyl-3-quinolin-2-yl-methyl-indol-1-yl)acetic acid.

In a similar manner to Example 1, except that the reaction was performed in a total of 12 relative volumes of water and the temperature was adjusted to 50±3° C. following acidification, 65 g of 5-fluoro-2-methyl-3-quinolin-2-yl-methyl-indol-1-yl)acetic acid was agglomerated over a period of 7.5 hours at the stated temperature to provide 59.5 g of product (92% yield, uncorrected for assay).

### Example 3

### Method for Preparing Spherical Agglomerates of Timapiprant by Re-processing of 5-fluoro-2-methyl-3-quinolin-2-yl-methyl-indol-1-yl)acetic Acid (Large Scale Example)

5-Fluoro-2-methyl-3-quinolin-2-yl-methyl-indol-1-yl)acetic acid (70.0 kg) was added to a stirred mixture of 50% w/w potassium hydroxide (45.1 kg, 22.6 kg active, 2 mol eq.), purified water (596 kg) and ethanol (9.3 kg) then the suspension was heated to 60±5° C. and held at this temperature for 30 minutes when most of the solid had dissolved.

The resulting cloudy solution was polish filtered at 60±5° C. and the equipment was rinsed with purified water (70 kg) at 60±5° C. Filtered 80% formic acid (34.7 kg, 27.8 kg active, 3 mol eq.) was added to the combined filtrates over a 3 hour period at 60±5° C., adjusting the stirring speed as necessary to maintain flowability of the mixture, and was followed by a line rinse of purified water (36 kg). After holding the resulting suspension at 60±5° C. for 45 minutes and then cooling to 30±3° C., filtered toluene (42 kg) was added over 1 hour at 30±3° C. The mixture was then held at this temperature for 14 hours and progress of the agglomeration monitored by in-line FBRM analysis.

The solid was collected by filtration, washed with purified water (9×140 kg) at 25±10° C. and then dried under vacuum at 70° C. maximum. The dried product was passed through a mechanical sieve and homogenized to provide 66.3 kg of (5-fluoro-2-methyl-3-quinolin-2-yl-methyl-indol-1-yl)acetic acid as a yellow solid (95% yield, uncorrected for assay).

### Example 4

### Method for Preparing Spherical Agglomerates of Timapiprant Starting from 5-fluoro-2-methyl-3-quinolin-2-yl-methyl-indol-1-yl)acetic Acid, Ethyl Ester

A stirred suspension of 5-fluoro-2-methyl-3-quinolin-2-yl-methyl-indol-1-yl)acetic acid, ethyl ester (30 g) in a mixture of 50% w/w aqueous potassium hydroxide (19.3 g, 9.7 g active, 2.2 mol eq.) and purified water (255 ml) was heated to 60±5° C. and held at this temperature for 10 hours then cooled to 20° C. and held overnight. After re-heating to 60±5° C., the resulting solution was polish filtered and the equipment rinsed through with purified water (30 ml). A solution of 80% formic acid (14.9 g, 11.9 g active, 3.25 mol eq.) was added to the combined filtrates over a 3 hour period at 60±5° C. followed by a purified water rinse (15 ml). After holding the resulting suspension for 30 minutes at the same temperature, the mixture was then cooled to 30±3° C. Toluene (17.9 g) was added over 1 hour at 30±3° C. and the mixture was then held at this temperature for 18 hours to allow agglomeration. The solid was collected by filtration, washed with purified water (9×60 ml) and then dried under vacuum at 70° C. to provide (5-fluoro-2-methyl-3-quinolin-2-yl-methyl-indol-1-yl)acetic acid as a yellow solid (27.3 g, 91% yield uncorrected for assay).

### Example 5

### Method for Preparing Spherical Agglomerates of Timapiprant Starting from 5-fluoro-2-methyl-3-quinolin-2-yl-methyl-indol-1-yl)acetic Acid, Ethyl Ester (Large Scale Example)

5-Fluoro-2-methyl-3-quinolin-2-yl-methyl-indol-1-yl)acetic acid, ethyl ester (80.0 kg) was added to a stirred mixture of 50% w/w potassium hydroxide (47.6 kg, 23.8 kg active, 2 mol eq.) and purified water (633 kg) then the suspension was heated to 60±5° C. and held at this temperature for 8 hours. The resulting solution was polish filtered at 60±5° C. and the equipment was rinsed through with purified water (73 kg) at 60±5° C. Filtered 80% formic acid (36.7 kg, 29.4 kg active, 3 mol eq.) was added to the combined filtrates over a 3 hour period at 60±5° C., adjusting the stirring speed as necessary to maintain flowability of the mixture, and was followed by a line rinse of purified water (41 kg). After holding the suspension at 60±5° C. for 1 hour and then cooling to 30±3° C., filtered toluene (44.8 kg) was added over 1 hour at 30±3° C. The mixture was then held at this temperature for 12 hours and progress of the agglomeration monitored by in-line FBRM analysis. The solid was collected by filtration, washed with purified water (9×160 kg) at 25±10° C. and then dried under vacuum at 70° C. maximum. The dried product was passed through a mechanical sieve and homogenized to provide (5-fluoro-2-methyl-3-quinolin-2-yl-methyl-indol-1-yl)acetic acid as a yellow solid (69.7 kg, 94% yield uncorrected for assay).

### Example 6

### Measurements of Primary Particle Size of Timapiprant Obtained After the Break Up of Spherical Agglomerates

The primary particle size distribution results are produced by means of a Malvern 2000 laser diffraction equipment by the analysis of the primary particles in suspension.

The particle size distribution laser diffraction measures were performed using a Malvern Mastersizer 2000 equipped with a Hydro 2000S cell for the wet suspension of the Timapiprant primary particles. The spherical agglomerates obtained according to the Examples 3 and 5, were dispersed in water by means of 10 minutes sonication and then added to the cell, previously filled with water, until an obscuration within 5-10% was obtained. The stirring rate was set at 1500 rpm and each of the suspensions produced were analysed by 10 acquisitions, the results are reported in Tables 1 and 2.

The Table 1 shows the PSD results by laser diffraction of Timapiprant primary particles obtained by means of spherical agglomeration process of Example 3.

TABLE 1
PSD

| Measurement | d (0.1) | d (0.5) |
|---|---|---|
| Measure 1 | 0.28 | 0.561 |
| Measure 2 | 0.278 | 0.559 |
| Measure 3 | 0.28 | 0.561 |
| Measure 4 | 0.279 | 0.56 |
| Measure 5 | 0.275 | 0.555 |
| Measure 6 | 0.28 | 0.56 |
| Measure 7 | 0.275 | 0.555 |
| Measure 8 | 0.28 | 0.56 |
| Measure 9 | 0.28 | 0.561 |
| Measure 10 | 0.28 | 0.56 |
| Mean result | 0.28 | 0.56 |

The PSD curve of timapiprant primary particles size obtained by means of spherical agglomeration process of Example 3 is showed in FIG. 4.

The Table 2 shows the PSD results by laser diffraction of Timapiprant primary particles size obtained by means of spherical agglomeration process of Example 5.

TABLE 2
PSD

| Measurement | d (0.1) | d (0.5) |
|---|---|---|
| Measure 1 | 0.315 | 0.64 |
| Measure 2 | 0.315 | 0.64 |
| Measure 3 | 0.315 | 0.64 |
| Measure 4 | 0.314 | 0.639 |
| Measure 5 | 0.314 | 0.64 |
| Measure 6 | 0.314 | 0.64 |
| Measure 7 | 0.314 | 0.642 |
| Measure 8 | 0.314 | 0.638 |
| Measure 9 | 0.314 | 0.64 |
| Measure 10 | 0.314 | 0.641 |
| Mean result | 0.31 | 0.64 |

The PSD distribution curve of timapiprant primary particles size obtained by means of spherical agglomeration process of Example 5 is showed in FIG. 5.

### Example 7

### Method for Preparing Spherical Agglomerates of timapiprant by Re-processing of 5-fluoro-2-methyl-3-quinolin-2-yl-methyl-indol-1-yl)acetic Acid (Large Scale Example).

5-Fluoro-2-methyl-3-quinolin-2-yl-methyl-indol-1-yl)acetic acid (65.8 kg) was added to a stirred mixture of 50% w/w potassium hydroxide (42.4 kg, 21.2 kg active, 2 mol eq.), purified water (659 kg) and ethanol (8.7 kg) then the suspension was heated to 60±5° C. and held at this temperature for 30 minutes when most of the solid had dissolved.

The resulting cloudy solution was polish filtered at 60±5° C. and the equipment was rinsed with purified water (66 kg) at 60±5° C. Filtered 80% formic acid (32.6 kg, 26.1 kg active, 3 mol eq.) was added to the combined filtrates over a 3 hour period at 60±5° C., adjusting the stirring speed as necessary to maintain flowability of the mixture, and was followed by a line rinse of purified water (33 kg). After holding the resulting suspension at 60±5° C. for 30 minutes and then cooling to 30±3° C., filtered toluene (39.5 kg) was added over 1 hour at 30±3° C. The mixture was then held at this temperature for 13 hours and progress of the agglomeration monitored by in-line FBRM analysis.

The solid was collected by filtration, washed with purified water (6×132 kg) and methyl tent-butyl ether (4×98 kg) at 25±10° C. and then dried under vacuum at 70° C. maximum. The dried product was passed through a mechanical sieve and homogenized to provide 60.3 kg of (5-fluoro-2-methyl-3-quinolin-2-yl-methyl-indol-1-yl)acetic acid as a yellow solid (92% yield, uncorrected for assay).

### Example 8

### Preparation of Tablets Comprising Spherical Agglomerates of Timapiprant in Comparison to Micronized Timapiprant

The spherical agglomerates of timapiprnat obtained according to Example 5, and the micronized timapiprant obtained as above described, are used to prepare tablets having the composition of Table 3 below.

The micronized timapiprant is obtained isolating timapiprant after synthesis by acidification with HCl of the potassium salt at pH 5.5-6.0 from a THF/water mixture. The crude was re-dissolved in formic acid and added to water in order to cause product precipitation. Upon isolation by filtration and drying at 70° C., the timapiprant was micronized according to the known technique, e.g. Rasenack et al. Micron-size drug particles: common and novel micronization techniques Pharm Dev Technol. 2004;9(1): 1-13.

TABLE 3
Tablets composition

| | Tablet A | Tablet B | |
|---|---|---|---|
| | per tablet | per tablet | |
| Components | [mg] | [mg] | [%] |
| Spherical agglomerates of | 50.00 | | 20.00 |
| Timapirant | | | |
| Timapirprant micronized | | 50.00 | 20.00 |
| Lactose monohydrate DC | 103.75 | 103.75 | 51.50 |
| Microcrystalline cellulose | 75.00 | 75.00 | 30.00 |
| Croscarmellose sodium | 7.50 | 7.50 | 3.00 |
| Poloxamer | 7.50 | 7.50 | 3.00 |
| Sodium lauryl sulphate | 2.50 | 2.50 | 1.00 |
| Colloidal Silicium dioxide | 1.25 | 1.25 | 0.50 |
| Magnesium stearate | 2.50 | 2.50 | 1.00 |
| Total | 250.00 mg | 250.00 mg | 100.00% |

The spherical agglomerates of timapiprant or the micronized timapiprant, together with the microcrystalline cellulose, the croscarmellose sodium, the poloxamer, the sodium lauryl sulphate and the colloidal silicon dioxide in the above amounts, are sieved in a sieve of 1.2 mm. The lactose monohydrate is then added, and the resulting mixture is blended in a free fall blender for at least 15 minutes. The magnesium stearate is added to the obtained mixture which is further blended in the free fall blender for additional 3 minutes.

The characteristics of the obtained blend material in terms of flowability are reported in Table 4.

TABLE 4
Flowability

| | Spherical | |
|---|---|---|
| | agglomerates of | Micronized |
| API | timapirpant | timapiprant |
| Appearance | Yellowish powder | Yellowish powder |
| Flowability (s/100 g) | 4 sec | ∞ |

The blend material is then compressed to obtain tablets, in a Tabletting machine Korsch XL 100 equipped with 8.0 mm punches, applying the following compression parameters of Tables 5 and 6:

TABLE 5
Tablet A (spherical agglomerates according to the invention)

| | Start of | Middle of | End of |
|---|---|---|---|
| | compression | compression | compression |
| Average weight | 251.71 mg | 250.17 mg | 248.3 mg |
| Individual | 248.7-255.3 mg | 247.5-253.5 mg | 241.3-251.5 mg |
| weights ± 3% | | | |
| Thickness | 4.47 mm | 4.44 mm | 4.43 mm |
| | (4.44-4.50) | (4.43-4.45) | (4.40-4.45) |
| Diameter | 8.06 mm | 8.06 mm | 8.07 mm |
| | (8.04-8.09) | (8.04-8.09) | (8.04-8.13) |
| Hardness | 120.1N | 119.1N | 109.2N |
| | (102-133) | (107-127) | (104-120) |

TABLE 6
Tablet B (micronized timapiprant)

| | Start of | Middle of | End of |
|---|---|---|---|
| | compression | compression | compression |
| Average weight | 6.4 mg | n.d | n.d |
| Individual | 228.4-265.6 mg | n.d. | n.d. |
| weights ± 3% | | | |
| Thickness | 4.36 mm | n.d. | n.d. |
| | (4.3-4.46) | | |
| Diameter | 8.06 mm | n.d | n.d |
| | (8.04-8.08) | | |
| Hardness | 96.10N | n.d | n.d |
| | (72-159) | | |

As can be appreciated in Table 4, the blend material comprising spherical agglomerates of timapiprant has shown a good flowability and it is suitable to be directly compressed to obtain tablets.

As shown in Table 6, the blend comprising micronized timapiprant was not suitable to be compressed with the tabletting machine, because the blend showed strong adhesion to the walls of the powder funnel. This led to very poor flow properties and made it was impossible to achieve the target tablet weight and hardness.

### Example 9

### Dissolution Profile of Tablets Comprising Timapiprant Agglomerates and Micronized Timapiprant

The dissolution profiles of tablets comprising spherical agglomerates of timapiprant (Tablets A) and tablets comprising micronized timapiprant (Tablets B) have been measured.

In order to obtain suitable tablets comprising micronized timapiprant for dissolution measurements, the tablets were compressed by hand. The timapiprant micronized and the excipients as reported in Table 3, were weighted and filled into the die and compressed by manually operating the machine.

The dissolution test was performed in Paddle Type Apparatus II according to USP <711>. The dissolution parameters are reported in Table 7.

TABLE 7
Dissolution parameters

| Dissolution parameters | Values |
|---|---|
| Dissolution medium | 50 mM NaH₂PO₄ H₂O buffer |
| | pH 6.8 + 0.4% w/v CTAB |
| Dissolution medium volume | 900 mL |
| Dissolution medium temper- | 37° C. ± 0.5° C. |
| ature | |
| Rotation speed | 75 rpm |
| Sampling time | 30 minutes for specification point |
| | determination or additionally 5, 15, 45 |
| | and 60 minutes for a profile. |
| | Different or additional time points are |
| | permitted as needed for a desired |
| | profile, subject to client preapproval. |
| Number of units tested | According to the acceptance table of USP |
| | <711>, minimum of 6, 1 per vessel). |
| Test solution/sampling | Withdraw using a glass pipette 5.0 ml of |
| | test solution at specific time point (30 |
| | min.) or, for determining dissolution |
| | profiles, at 5, 15, 30, 45 and 60 min, and |
| | filter the sample through a 0.45 μm nylon |
| | syringe filter, discard the first 2 mL |
| | of filtrate. |

The collected samples are analysed in chromatography using a column Waters Symmetry Shield RPB, 75×4.6 mm, 3.5 μm or equivalent, according to any method known in the art.

The comparison of dissolution profiles of Tablets A and Tablets B are reported in the Table 8 and the curves are represent in FIG. 11.

TABLE 8
Dissolution profile

time in minutes

| | 0 | 5 | 15 | 30 | 45 | 60 |
|---|---|---|---|---|---|---|
| Tablets A | 0 | 75% | 89% | 94% | 95% | 96% |
| Tablets B | 0 | 80% | 90% | 92% | 92% | 92% |

As can be appreciated from the Table 8 and FIG. 11, the dissolution profiles of Tablets A and B are analogues, demonstrating that even if Tablets A comprise spherical agglomerates of timapiprant having a greater particle size respect to the micronized timapiprant, the Tablets A are provided with a proper dissolution profile as required for a poorly soluble active ingredient such as timapiprant.

### Example 10

### Measurements of Specific Surface Area (SSA) of Spherical Agglomerates of Timpapiprant and Micronized Timapiprant

The specific surface areas (SSA) of spherical agglomerates of timapiprant obtained according to Examples 3, 5,7 and the micronized timapiprant were measured was determined by BET nitrogen adsorption using the TriStar of Micromeritics apparatus according to following parameters:
Isotherm, Adsorbing/Desorbing Run: 0 to 1 p/p0
Vials: ⅜ Inch, Degassing Pre-analysis: 2 hours at 40° C., Nitrogen as adsorbate
Preparation: Evacuation Rate 10 mmHg/s; Unrestricted evacuation from 5 mmHg
Vacuum setpoint: 10 mmHg, Evacuation time: 1 hour
Leak Test Duration: 120 s, Outgas Test Duration: 180 s
Measure p0 at intervals during the analysis, Enter the analysis bath temperature: 120 min./77 kelvin
Equilibration Interval: 5 s; Minimum Equilibration Delay at p/p0≥0,995 per 600 s.

The Table 9 shows the specific surface area values of spherical agglomerates of timapiprant and the micronized timapiprant.

TABLE 9
Specific surface area

| API | SSA (m²/g) |
|---|---|
| Spherical agglomerates of | 13.1 |
| timapiprant of Example 3 | |
| Spherical agglomerates of | 10.1 |
| timapiprant of Example 5 | |
| Spherical agglomerates of | 15.5 |
| timapiprant of Example 7 | |
| Micronized Timapiprant | 8.0 |
| Micronized Timapiprant | 7.8 |

1. A pharmaceutical composition in a solid form comprising spherical agglomerates of timapiprant, wherein said spherical agglomerates of timapiprant are obtained by a process comprising the steps of:
a) preparing an aqueous suspension of 5-fluoro-2-methyl-3-(quinolin-2-ylmethyl)-1H-indol-1-yl)acetic acid of formula I; 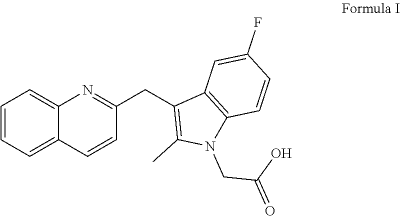 [SMILES: Cc1c(Cc2ccc3ccccc3n2)c2cc(F)ccc2n1CC(=O)O]
b) agglomerating the particles of 5-fluoro-2-methyl-3-(quinolin-2-ylmethyl)-1H-indol-1-yl)acetic acid obtained in step a) by addition of an agglomerating agent;
c) isolating the agglomerates obtained in step b); and optionally
d) washing and drying the isolated agglomerates.

2. The pharmaceutical composition according to claim 1, wherein said spherical agglomerates of timapiprant have a particle size greater than 3 μm.

3. The pharmaceutical composition according to claim 2, wherein said spherical agglomerates of timapiprant have a particle size between 10 and 300 μm.

4. The pharmaceutical composition according to claim 3, wherein said spherical agglomerates of timapiprant have a particle size between 10 and 200 μm.

5. The pharmaceutical composition according to claim 4, wherein said spherical agglomerates of timapiprant have a particle size between 10 and 50 μm.

6. The pharmaceutical composition according to claim 1, wherein said spherical agglomerates of timapiprant have a specific surface area determined by BET nitrogen adsorption of greater than 9 m²/g.

7. The pharmaceutical composition according to claim 1, further comprising pharmaceutical acceptable excipients or carriers.

8. The pharmaceutical composition according to claim 1, wherein the composition is for oral administration.

9. The pharmaceutical composition according to claim 1, wherein the solid form is a tablet.

10. The pharmaceutical composition according to claim 1, wherein the amount of timapiprant is between 5 mg and 100 mg per unit.

11. The pharmaceutical composition according to claim 10, wherein the amount of timapiprant is between 10 mg and 50 mg per unit.

12. The pharmaceutical composition according to claim 9, wherein the composition releases at least 75% of the timapiprant at 5 minutes and 90% of the timapiprant at 60 minutes when tested in a Paddle Type-II dissolution apparatus according to USP <711>, in 900 mL of 50 mM monosodium phosphate buffer, at a paddle speed of 75 RPM and at a temperature of 37° C.

13. A process for the preparation of the pharmaceutical composition according to claim 1, comprising the steps of:
a) preparing an aqueous suspension of 5-fluoro-2-methyl-3-(quinolin-2-ylmethyl)-1H-indol-1-yl)acetic acid of formula I; 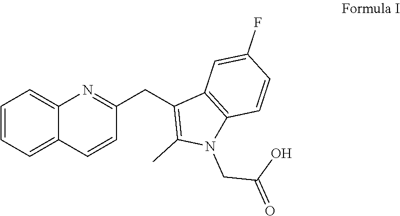 [SMILES: Cc1c(Cc2ccc3ccccc3n2)c2cc(F)ccc2n1CC(=O)O]
b) agglomerating the particles of 5-fluoro-2-methyl-3-(quinolin-2-ylmethyl)-1H-indol-1-yl)acetic acid obtained in step a) by addition of an agglomerating agent;
c) isolating the agglomerates obtained in step b);
d) washing and drying the isolated agglomerates;
e) mixing the isolated agglomerates obtained in step d) with pharmaceutical acceptable excipients;
f) compressing the mixture obtained in step e) to obtain tablets.

14. The process for the preparation of the pharmaceutical composition according to claim 13, further comprising a step of granulation of the mixture obtained in step e).

15. The process according to claim 13, further comprising a step of granulation of the isolated agglomerates obtained in step d).

16. A method of treating respiratory diseases in a subject in need thereof, comprising administering to the subject a pharmaceutical composition according to claim 1.
